(19) United States
(12) Patent Application Publication
(10) Pub. No.: US 2003/0216701 A1
(43) Pub. Date: Nov. 20, 2003

(54) Synthetic glove and process for making

(76) Inventor: Gentho Sumarta, Jakarta (ID)

Correspondence Address: GOODWIN PROCTER L.L.P., 7 BECKER FARM ROAD, ROSELAND, NJ, 07068, US

(21) Appl. No.: 10/323,394

(22) Filed: Dec. 17, 2002

(30) Foreign Application Priority Data
May 15, 2002 (ID) P00200200273

(51) Int. Cl. A61M 35/00; B28B 1/38

(52) U.S. Cl. 604/290; 264/301

(57) ABSTRACT

A process for preparing a synthetic, individual latex glove including mixing step the base material of glove comprising PVC resin, or latex, plasticizer, stabilizer and then added with vitamin E. The preparation including dipping of glove mold into the dipping tank, heating, cooling, forming, applying powder or liquid in glove and removing the glove.

### FIELD OF INVENTION

The present invention relates to synthetic gloves made with added vitamin E (alpha-tocopherol).

### BACKGROUND OF THE INVENTION

The conventional synthetic clove (including in this term gloves from synthetic resins, such as PVC, as well as of latex) is composed of the thin latex or synthetic film or rubber designed fit tightly around the hand, thereby will give comfort sensitivity to the hand.

The glove functions as a protector for preventing contamination from foreign materials of the skin of the hand of the wearer. Therefore the skin of the hand that will be in direct contact with the glove should be protected form adverse effects, and to minimize such effects.

### SUMMARY OF THE INVENTION

The present invention is a synthetic glove made of PVC or latex added with vitamin E (alpha-tocopherol) as agent such an agent which would be able to maintain rejuvenation of skin.

Therefore, it is a principal objective of the present invention to provide a preparation of synthetic glove containing vitamin E (alpha-tocopherol).

A synthetic glove composition accompanied with vitamin E suitably contains (based on parts by weight):

| | |
|---|---|
| PVC resin or latex | 100 |
| Plasticizer | 20-80 |
| Stabilizer | 0.5-2 |
| Vitamin E | 0.01-0.2 |

The thickness of the glove suitably ranges between 0.08 mm. To 0.15 mm. A further object of the present invention is to provide a mixing process for material to be used in preparing the glove. The mixing process includes the following steps:

1. Mixing plasticizer, stabilizer and PVC resin, at between 900 to 1000 rpm until temperature reaches 44°±1° C.;

2. Adding vitamin E after step (1) and continue mixing by speed between 9000-1000 rpm, until the temperature reaches 47° C.

Further process steps of forming the glove, include:

1. Dipping the glove mold into material produced form the aforesaid mixing process;

2. Heating said dipped glove mold to a temperature between 200° and 300° for 12±2 minutes;

3. Cooling the glove by fans;

4. Forming a lip;

5. Applying a powder/liquid into the glove; and

6. Removing the produced glove.

### BRIEF DESCRIPTION OF THE DRAWINGS

The invention is described in further detail, with reference being had to the drawing, wherein:

FIG. 1 is a flow diagram of mixing process to be used in preparing glove; and

FIG. 2 is a production process plot of glove according to the present invention.

### DETAILED DESCRIPTION

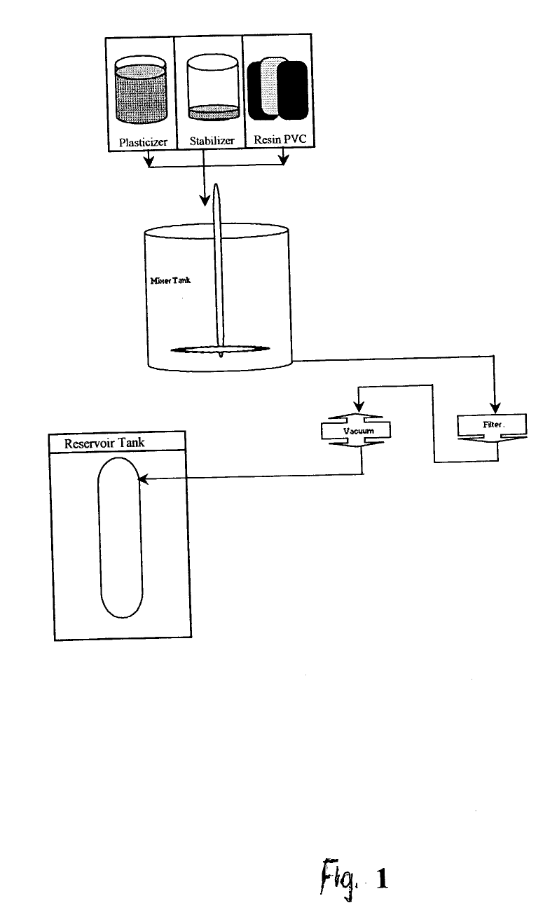

FIG. 1, showing a flow diagram of the process of mixing in preparing the material for the glove, of e.g. PVC resin, plasticizer, stabilizer and accompanied by adding vitamin. First, the plasticizer, stabilizer and PVC resin are introduced into the mixer tank. Mixing is carried out by stirring at a speed between 900 and 100 rpm, until the temperature reaches 44°±1° C. Then, vitamin E is added to the mixture and the mixing is continued until the temperature reaches 47° C. After that the mixture is transferred into a storage tank by a pump and through a filter. The mixture is incubated for at least 12 hours, before further processing.

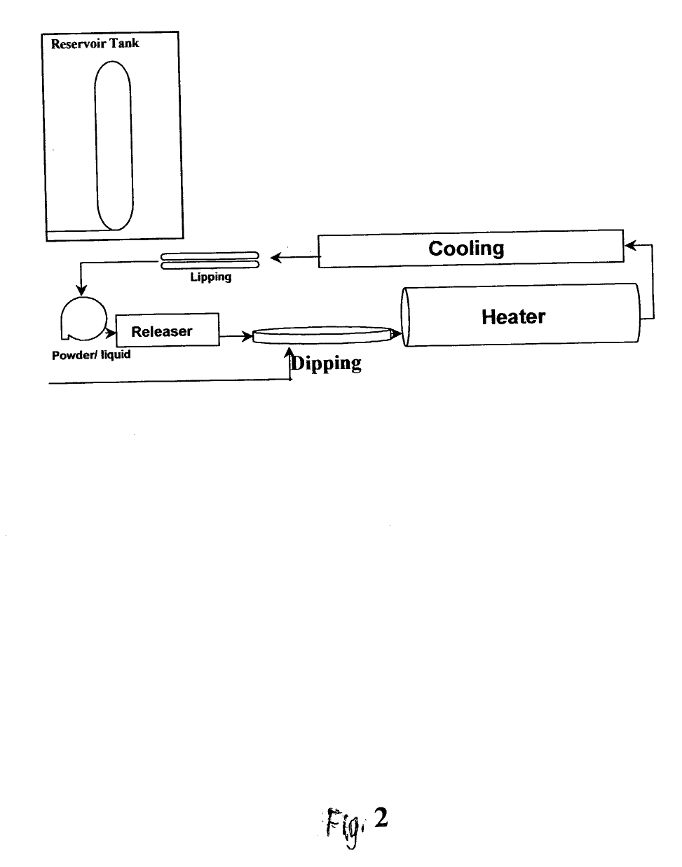

FIG. 2, shows a flow diagram of the glove production process. The first, make condition of the glove making machine until heater reach between 200 to 350° C. Then, dipping the glove mold in the dipping tank which previously have been filled material of the mixture product mentioned above, afterwards heated in oven in a temperature between 200° to 350° for 12±2 minutes. After that, the cooling step is carried out by using several fans. After passed the cooling process then coming in lipping forming and continued by giving donning coating and finally until the glove removing process.

Specification of the produced glove is as follows:

| | |
|---|---|
| Thickness | 0.08 to 0.15 mm |
| Weight | 7 to 10 gr per glove depending on size of glove |
| Length | 26 to 30 cm |
| Width | 80 to 121 mm (depending on size of glove) |

As is understood from the aforementioned described embodiments, that the present invention of synthetic glove made of PVC resin by added vitamin E-alpha-tocopherol will give more advantage for user, that is skin of hand will maintain its rejuvenation so that user will feel more comfort, safe and more confident, because presence of the protection in the hand skin about as result of direct contact between skin of hand with glove. This case caused by effect of vitamin E-alpha-tocopherol showed in chemical structure work mechanism below.

1. Synthetic glove containing vitamin, with the composition (parts by weight):

| | |
|---|---|
| PVC resin or latex | 100 |
| plasticizer | 20-80 |
| stabilizer | 0.5-2 |
| vitamin E-alpha-tocopherol | 0.01-0.02 |

2. The glove in claim 1, having a thickness of from 0.08 to 0.15 mm.

3. A process for preparing a synthetic glove material containing vitamin E which comprises:
1. dipping the glove mold into material produced form the mixing process of claim 3;
2. heating the mold to a temperature between 200° and 300° for 12±2 minutes;
3. cooling glove by a fan;
4. forming a lip;
5. applying powder or liquid to the glove; and
6. removing the produced glove.
